(19) United States
(12) Patent Application Publication
(10) Pub. No.: US 2006/0083732 A1
(43) Pub. Date: Apr. 20, 2006

(54) Hyaluronic acid in the enhancement of lens regeneration

(76) Inventor: Arlene Gwon, Newport Beach, CA (US)

Correspondence Address: Advanced Medical Optics, Inc.;Legal Department, 1700 E. St. Andrew Place, Santa Ana, CA, 92705, US

(21) Appl. No.: 11/293,682

(22) Filed: Dec. 1, 2005

Related U.S. Application Data
(63) Continuation-in-part of application No. 10/881,426, filed on Jun. 30, 2004.

(51) Int. Cl. A61K 38/47; A61K 31/728

(52) U.S. Cl. 424/94.61; 514/54

(57) ABSTRACT

The present invention addresses the treatment of ocular conditions by the enhancement of lens regeneration. This is accomplished by the administration of a high viscosity composition including a hyaluronic acid compound. Excess high viscosity composition may be removed by focal laser photophacoablation or through the use of a digestive compound or enzyme such as hyaluronidase. Additionally, a collagen product may be injected within the lens capsule to improve lens cell proliferation and differentiation, and to improve the configuration, shape and structure of regenerated lenses. Various embodiments involving the enhancement of lens regeneration are described. For example, lens regeneration may be enhanced by filling the lens capsule bag with the inventive hyaluronic acid compound; by inserting at least one collagen patch in the lens capsule; and/or by injecting a collagen-based product into the lens capsule.

### FIELD OF THE INVENTION

The present invention relates to the nanotechnology of tissue engineered optical systems, and more specifically, the regeneration of ocular tissue and related methods to treat ocular conditions, as for example, lenticular disorders.

### BACKGROUND OF THE INVENTION

Regeneration and repair are the fundamental features of the healing response. The ability to regenerate (i.e., to replace damaged tissue with healthy cells of similar type) varies among tissues and may be seen in the corneal epithelium and conjunctiva. While the healing response in and around the eye occurs primarily because of tissue repair mechanisms (i.e., damaged tissue is replaced by a newly generated fibrous connective tissue) rather than regeneration, there is substantial data suggesting that regeneration of the natural lens is possible. Ideally, the regenerated lens, with or without a suitably flexible and biocompatible polymeric lens, would have all the properties of the natural lens including clarity, protein content, histology, focusing power and accommodative ability. Optimally, corrective powers could optionally be included, later added, in combination with related mechanisms imparting ameliorative visual acuity enhancements.

Extracapsular cataract extraction with implantation of an intraocular lens (IOL) is currently the most common method for the treatment of cataracts. This procedure is less than ideal because the current synthetic intraocular lenses are unable to accommodate appreciably, and secondary opacification of the posterior capsule is a common occurrence. While intraocular implantation of multifocal or accommodating intraocular lenses (IOLs) attempt to address the need for far and near vision in the cataract patient, they are complicated by the development of posterior capsule opacification and visual dysphotopsias. Posterior capsule opacification (PCO) occurs secondary to anterior lens epithelial cell migration and myoblastic transformation and contributes to wrinkling of the posterior capsule and visual distortion.

Ideally, if a regenerated natural lens could replace a suitable biodegradeable material, the reformed lens would have the same or similar natural focusing power as the normal young lens and be able to accommodate. Alternatively, if naturally regenerating lens epithelial cells could be directed to grow in a regularly organized pattern around a suitably flexible and biocompatible polymeric lens, the resultant bilenticular system might be able to accommodate. Other and further corrections and enhancements would be within the purview of artisans, and are within the scope of the instant teachings.

Hyaluronic acid has been shown to be beneficial in wound healing in various body tissues. Hyaluronic acid in the form of Healon® ophthalmic viscoelastic solution (“OVD”) (available from Advanced Medical Optics Gronigen BV, Gronigen, NL) has been used to fill the lens capsule bag following phacoemulsification (i.e., a cataract surgical procedure which uses an ultrasonic vibration to shatter and break up a cataract for removal) and irrigation/aspiration of both the natural and cataractous lens and sealing of the anterior capsule in the rabbit. However, the Healon® OVD normally is resorbed by about one week postoperatively when the regenerating lens cells are in various stages of development. Additionally, over time the regenerated lens has had an abnormal nucleus in the form of a star-shaped opacity as the earliest lens fibers regenerated at different rates.

There is therefore a need in the art for a regenerated lens (with or without a suitably flexible and biocompatible polymeric lens) which would have all the properties of the natural lens including clarity, protein content, histology, focusing power, accommodative ability, configuration, shape and structure. There is a further need in the art for the regeneration of a clear natural lens with or without a biocompatible polymer lens in which the former may be applicable to treatment of cataract in the pediatric population and the latter suitable for adult cataract, offering true accommodation and correction of presbyopia. There is additionally a need to improve lens cell proliferation and differentiation following phacoemuslification and irrigation/aspiration. Furthermore, there is a need in the art to treat ocular disease and/or correct vision impairments without its associated complications, as for example, posterior capsule opacification.

### BRIEF DESCRIPTION OF THE FIGURES

FIG. 1 depicts a digital image analysis graph of lens regrowth rates in old rabbits (Group 1), young rabbits (Group 2), and young rabbits with low vacuum of capsule bag (Group 3) in accordance with an embodiment of the present invention.

FIGS. 2A and 2B illustrate representative scanning electron micrographs of fibers formed around an intralenticular implant in accordance with an embodiment of the present invention.

FIG. 3 (prior art) illustrates the gross morphology of the lens fibers generated in the cortex in accordance with an embodiment of the present invention.

FIG. 4 (prior art) illustrates a lens fiber depicted in FIG. 3 in accordance with an embodiment of the present invention.

FIG. 5 depicts the honeycomb appearance of Perlane® OVD in the lens capsule bag in accordance with an embodiment of the present invention.

FIG. 6 depicts the clarity of the regenerated lens structure adjacent to the Perlane® OVD in accordance with an embodiment of the present invention.

FIG. 7 depicts the honeycomb appearance of the early lens growth enhanced by Restylane® OVD in accordance with an embodiment of the present invention.

FIG. 8 depicts the honeycomb appearance of the early lens growth enhanced by Restylane® OVD in accordance with an embodiment of the present invention.

FIG. 9 depicts the initial cobblestone (hexagonal) appearance of Restylane® OVD.

FIG. 10 depicts an eye one day following injection of Vitrase, where the Restylane® OVD appears to be dissolved and a clear fluid filled area may be seen in the center of the regenerating lens.

FIG. 11 depicts a spherical regenerated lens from the one week group with a cortex having a good structure. A few vacuoles of retained Restylane OVD and a lacy nuclear star opacity may be seen.

FIG. 12 a spherical regenerated lens from the one month group, showing a cortex having good structure and the capsulotomy sealed to linear scar.

### DETAILED DESCRIPTION

The present invention is based on the concept that the natural lens is capable of controlled or enhanced organic cellular or biological regeneration following endocapsular lens and/or cataract extraction. In various embodiments, the present invention provides methods to produce a regenerated lens with properties similar to that of the natural lens, including clarity, protein content, histology, focusing power and accommodative ability. In one embodiment, the natural regenerating lens tissue may be directed to grow in a more natural or regular pattern around a suitably flexible and biocompatible polymeric lens. The resultant bilenticular system of this embodiment has clarity, focusing power and accommodative ability similar to the natural lens. Yet, in another aspect, lenticular tissue may be engineered using focal laser photophacocoagulation to remove excess viscoelastic substances and/or to modify structure and clarity of the regenerated lens and/or bilenticular lens.

Lens regeneration is known to occur following endocapsular lens or cataract extraction in rabbits. In accordance with the present invention, the rate and quality of the regenerated lenses has been enhanced by sealing the capsulotomy and providing a hyaluronan-based viscoelastic as an internal scaffold for the proliferating lens epithelial cells left at the time of surgery. It has been shown that hyaluronic acid, for example Restylane OVD (Q-Med Aktiebolag, Sweden), appeared to enhance the alignment of the lens epithelium on the capsule prior to lens differentiation, thus promoting a more normal clarity and structure to the regenerated lens. However, it was also determined that the amount of lens regrowth was shown to be inversely related to the dose of Restylane OVD injected. Based on this finding, one aspect of the present invention teaches the administration of hyaluronidase to dissolve the retained hyaluronic acid following endocapsular lens extraction and lens regrowth.

FIG. 3 depicts the human lens and the gross morphology of the lens fibers generated in the cortex 15. FIG. 3 also depicts the capsule 10, nucleus 20, zonule 70, the anterior pole 30, equator 50 and optical axis 40 of the lens. These regenerated lens fibers originate as epithelial cells and elongate into ribbon-like nucleus-free lens components. The cross sections of these lens components are hexagonal in shape. FIG. 4 depicts a closer view of a lens fiber 80, which has a hexagonal cross-section.

Since the first description by Cocteau and Leroy D'Etoille (1827) the residual lens epithelial cells that contribute to secondary cataract formation have been shown to regenerate and differentiate more normally if the integrity of the lens capsule is restored following endocapsular lens extraction in rabbits. Lens fiber differentiation has been shown to follow a process similar to embryological development with cellular proliferation along the anterior and posterior capsule, followed by elongation of the posterior epithelial cells, anterior migration of fiber nuclei and subsequent differentiation at the equatorial zone. The regenerated lenses have been shown to contain all the major crystallins—alpha, beta and gamma—in proportions similar to fetal or normal lenses. In these earlier studies, regeneration is noted as early as 2-3 weeks postoperative, and capsule bag filling with regenerated lens tissue is seen at 7-10 weeks postoperative (A. Gwon et al., “Restoration of Lens Capsule Integrity Enhances Lens Regeneration in New Zealand Albino Rabbits,” ARVO, Sarasota, Fla. (May 1992)). In addition, lens regeneration has been shown to occur after endocapsular extraction of a concanavalin A-induced cataract.

As discussed herein, restoring the lens capsule integrity by insertion of a collagen patch at the time of surgery has enhanced the growth rate and shape/structure of the regenerated lenses in both rabbits and cats (Example 2). The regenerated lenses were spherical. Thus, the regenerated lenses had a normal cortex with good structure and clarity. The nucleus contained a star-shaped opacity related to the irregular growth pattern and misalignment of the earliest lens fibers. In previous studies, various viscoelastic agents had been used to fill the capsule bag following lens/cataract removal. Hyaluronic acid in the form of Healon® OVD has provided some success to date. However, hyaluronic acid in the commercial available form of Healon® OVD biodegrades too fast (this form of hyaluronic acid is resorbed by about one week postoperatively), and the regenerated lens has an abnormal nucleus in the form of a star-shaped opacity as the earliest lens fibers regenerate at different rates. If a cross-linked hyaluronic acid is used, the time required for the body to resorb the hyaluronic acid is significantly longer. Depending on the formulation, a cross-linked hyaluronic acid may not degrade at all. When a degradable cross-linked polymer is used, it may be used in conjunction with hyaluronidase to better regulate/control the timing and duration of the lens' exposure to hyaluronic acid, thereby modulating the growth process.

More recently, as the inventor has demonstrated, high viscosity hyaluronic acid provided an internal scaffold for the proliferation and differentiation of regenerating lens fibers following endocapsular lens extraction in Dutch Belt pigmented rabbits with good early lens fiber alignment and differentiation. The regenerated lenses have a normal spherical shape and the lens structure is clear with normal lens fiber alignment around the spherical residual viscoelastic material. In addition, in the one eye treated with focal photocoagulation with a Q-switched neodymium: YAG (Nd:Yag) laser, the inventor has demonstrated that partial clearing of the hyaluronic acid was attained. On histological examination, the lens structure was normal with a monolayer of anterior lens epithelium, lens differentiation occurring at the equatorial region and normal lens fiber structure. Centrally, the retained hyaluronic acid appears as an elliptical homogenous bluish mass.

The concept of creating a bilenticular system by implantation of a suitably flexible polymeric lens compatible with the naturally regenerating lens tissue was previously suggested by studies in which Acuvue® contact lenses (etafilcon A, 58% H₂O; available from Johnson & Johnson Vision Care, Inc., Jacksonville, Fla.) were modified for intralenticular implantation in the rabbit eye. While normal regeneration was noted in one eye, the results were inconsistent and the nucleus of most regenerated lenses contained a star-shaped opacity related to the irregular growth pattern and misalignment of the earliest lens fibers.

The mammalian lens, like other ectodermal tissues, can regenerate itself given the proper environment (A. Gwon et al., “Induction of de novo Synthesis of Crystalline Lenses in Aphakic Rabbits,” Exp Eye Res., 49:913-926 (1989)). Since 1781, researchers have known that the crystalline lens of amphibians can be regenerated after partial removal of the eye contents, or lensectomy. The lens is regenerated either from the corneal epithelium or the iris epithelim. Regeneration depends upon factors relating to the neural retina. Development of the new lens is somewhat different than development of the normal amphibian lens. In normal amphibian lens differentiation, gamma crystalline appears first, beta crystalline appears second, and alpha crystalline appears last. When the amphibian lens is regenerated from the iris epithelium, alpha and beta crystalline appear before gamma crystalline.

The ability to regenerate the crystalline lens appears to be lost in higher vertebrates. However, lens epithelial cells of birds and mammals can be grown in culture. Confluent monolayers (primary culture of chick lens epithelium) form masses of elongated cells, called lentoid bodies. In the chick, alpha, beta, and delta crystalline, as well as the main intrinsic membrane protein (MIP 26) are produced by the cells in these lentoid bodies. However, the relative proportions of these lens proteins do not resemble those present in normal chick lenses. Long-term culture of rabbit lens epithelial cells (primary culture in conditioned medium) has led to relatively stable cell lines containing the alpha crystalline promoter. These cell lines synthesize the A and B subunits of alpha crystalline. The beta-gamma crystalline family is not synthesized by these rabbit epithelial cells.

Differential crystalline synthesis is observed in cultured human epithelial cells. Cultures of human fetal lens epithelial cells express both the B chain of alpha-crystalline and one of the beta-Bp. Although human cell lines maintain their epithelial cell nature when grown on haptotactic surfaces, they form lentoid bodies on non-haptotactic surfaces. These lentoid bodies express gamma-crystalline.

With the advent of endocapsular phacoemulsification and posterior chamber phacoemulsification and aspiration after anterior lens capsule removal as treatment for cataracts, spontaneous growth has been observed to occur on that portion of the lens capsule remaining in the eye following surgery. Particular embodiments of the present invention are based on the inventor's study of the spontaneous growth in the lens capsules following endocapsular phacoemulsification.

The progressive steps in the process of spontaneous regeneration of tissue of ectodermal origin have been described well for the areas of skin epidermis and corneal epithelium. Another ectodermal derivative, the crystalline lens, was reported in 1827 to regenerate in rabbits. However, research in this area has progressed more slowly. Investigators have found that the lens regenerative process is dependent on an intact anterior and posterior lens capsule. After extraction of the lens capsular contents, regenerating lens tissue first is noted two weeks postoperatively, beginning in the periphery of the capsule and occurring more rapidly in younger rabbits.

In 1842, Valentin described for the first time the regenerated rabbit lens on a microscopic level, demonstrating the presence of characteristic round or polyhedral-shaped crystalline cells (G. Valentin, “Mikroscopische Untersuchung zweier wiedererzeugter Krystallinsen des Kaninchens,” Ztschr S. Rat. Med., 1, 227-37 (1842)). Valentin suggested that regeneration takes place by effusion into the capsule of initially liquid cytoblastic masses, which subsequently develop into lens cells and fibers. In 1960, Stewart showed that when embryonic tissue was implanted into the capsular bag after lens evacuation, the new lens fibers were aligned in the typical concentric pattern of the mature lens. Stewart also demonstrated that lens differentiation occurred at the equator (D. S. Stewart, “Further Observations on Degenerated Crystalline Lenses in Rabbits, with Special Reference to Their Refractive Qualities,” Trans Ophthalmol Soc UK, 80:357 (1960)).

To verify that the process develops from residual lens epithelial cells (rather than retained lens fibers), the inventor examined the histologic findings in the early post-operative period during the phase of early lens regrowth in rabbits.

Lens fiber differentiation in the embryo has been shown to involve loss of mitotic activity, marked cellular elongation, intensive synthesis of lens specific proteins called crystallines, and loss of the cell nucleus. As in embryonic development, lens regeneration proceeds by cellular proliferation along the anterior and posterior capsule (days 1-7) and is followed by elongation of the posterior epithelial cells and migration of the nuclei anteriorly (1 month). During the second month, lens differentiation occurs at the equatorial zone, with gradual elongation of cells, anterior migration of nuclei, and eventual loss of the nuclei. The mechanical forces exerted on the capsule bag may play an important role in lens fiber differentiation. The regenerative process in the New Zealand albino (NZA) rabbit after endocapsular lens extraction appears to follow the stages seen in the embryonic development of the lens (A. E. Gwon, et al., “A Histologic Study of Lens Regeneration in Aphakic Rabbits,” Investigative Ophthalmology & Visual Science, 31(3): 540-547 (1990)).

Extracapsular cataract extraction with intraocular lens (IOL) implantation is the procedure of choice for the treatment of cataracts. The single most frequent cause of decreased visual acuity after this surgery is delayed opacification of the posterior capsule. This opacification occurs secondarily to anterior lens epithelial cell migration and myoblastic transformation, contributing to wrinkling and fibrosis of the posterior capsule and resulting in visual distortion. IOL implantation tends to delay the onset of opacification.

Previous studies have shown that lens regeneration can occur spontaneously after endocapsular phacoemulsification and irrigation/aspiration of the lens capsular contents of NZA rabbits, when the anterior and posterior capsules are left relatively intact. The inventor has demonstrated that the growth curves for lens regeneration differ with age in NZA rabbits after endocapsular phacoemulsification of the lens and irrigation/aspiration. The inventor found that lens regeneration was significantly faster in younger animals (A. Gwon et al., “Lens Regeneration in Juvenile and Adult Rabbits Measured by Image Analysis,” Investigative Ophthalmology & Visual Science, 33(7):2279-2283 (1992)). Lens regeneration in young animals occurred as early as 2 weeks after surgery, and the capsular bag reached maximum filling capacity with newly regenerated lens material at approximately 3 months. In comparison, lens regeneration in adult animals was not observed until 5 weeks after surgery and was still occurring as long as 6 months later. A similar pattern occurs in humans. Posterior capsule opacification after extracapsular cataract extraction with IOL implantation occurs more frequently and at a much faster rate in children than in adults.

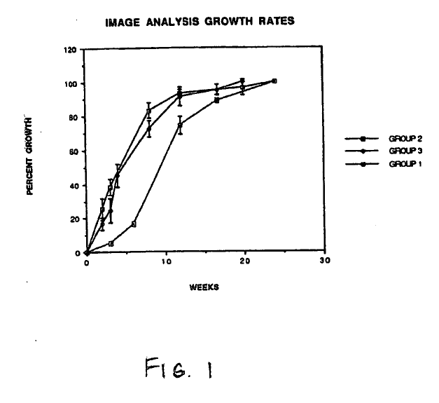

FIG. 1 depicts a digital image analysis graph of lens regrowth rates in old rabbits (Group 1), young rabbits (Group 2), and young rabbits with low vacuum of capsule bag (Group 3). As illustrated in FIG. 1, the initial lens regrowth rate in old rabbits was much slower than that of young rabbits.

The reported incidence of posterior capsule opacification (secondary cataract) varies greatly, depending upon patient age, follow-up time, and presence and type of IOL. In children, the incidence of posterior capsule opacification is nearly 100%, whereas in adults the reported incidence varies from 15% to nearly 50%, 2-3 years after surgery. Posterior capsule opacification is the product of the proliferation and migration of lens cells remaining in the capsular bag and the growth of cells of nonlenticular origin.

In another study, the inventor showed that lens regeneration was shown to occur after endocapsular extraction of posterior subcapsular cataracts induced by intravitreal injection of Concanavalin A (A. Gwon et al., “Lens Regeneration in New Zealand Albino Rabbits After Cataract Extraction,” Investigative Ophthalmology & Visual Science, 34(6): 2124-2129 (1993)). The regenerated lenses weighed less than lenses regenerated after normal lens extraction. The regenerated lenses of the cataract group were similar to the control normal lens group in transparency. The regenerated lenses are fairly translucent but because of abnormalities in the rate of regrowth in different parts of the capsule bag, these lenses are not optically clear and irregularities in structure exist. In addition, the lenses have varying degrees of vacuolization and some areas of opacification. No differences in structure and translucency of the regenerated lenses in the normal versus cataract lens group was visible by slit lamp biomicroscopy (not shown). However, the regenerated lenses were noted to be smaller.

In numerous studies, lenses that have been regenerated following endocapsular lens extraction in NZA rabbits have been irregular in shape, appearing primarily doughnut-shaped. The newly formed lenses are irregular in shape as a result of the lack of lens growth at the site of the anterior capsulotomy and its adhesion to the posterior capsule. These regenerated lenses have had variable translucency because of irregular alignment of newly formed fibers, which may partly result from irregular proliferation of cells in zones of wrinkling or folding of the lens capsule in the early postoperative period. To improve the transparency of the regenerated lenses and their therapeutic utility, investigators have attempted to mimic the embryonic environment with limited success.

It became apparent to the inventor that a suitable mechanism for sealing the anterior capsulotomy and restoring the continuity of the anterior capsule might be beneficial. Physical forces exerted on the lens may affect the rate of cell proliferation and distribution of dividing cells in this tissue. Accordingly, the inventor examined the enhancement of lens regeneration in NZA rabbits through the restoration of lens capsule integrity by sealing the anterior capsulotomy with a collagen patch and by filling the capsule bag with air, sodium hyaluronate (Healon® OVD), or perfluoropropane gas (A. Gwon et al., “Restoring Lens Capsule Integrity Enhances Lens Regeneration in New Zealand Albino Rabbits and Cats,” J Cataract Refract Surg., 19: 735-746 (1993)).

The inventor attempted to seal the anterior capsulotomy with fibrin sealant, Mussel adhesive protein, and cyanoacrylate. The inventor was able to restore the lens capsule integrity by inserting a collagen patch at the time of surgery to seal the capsule and restore its continuity and thus improve the shape and structure of the regenerated lens (Example 2). The inventor then filled the capsule bag with air, sodium hyaluronate (Healon® OVD), or perfluoropropane gas to prevent adhesions between the anterior and posterior capsules and to maintain capsule tautness and shape (Example 3).

Mayer showed that the process begins in the periphery of the capsule and progresses centrally toward the site of the anterior incision (Mayer, “Uber die reproduktion der Krystallinse,” Journal der Chirurgie und Augenheilkunde (Berlin, von Graefe und Walther) 17:524 (1832)). Textor found that lens regeneration was dependent on an intact anterior and posterior capsule and its form depended on the lesion of the capsule and how it had cicatrized (R. L. Randolph, “The Regeneration of the Crystalline Lens: An Experimental Study,” John Hopkins Hospital Reports, 9:237 (1900)). Sikharulidze and Stewart demonstrated that the rate and quality of the regenerated lens could be improved and had an “optical density similar to that of the normal crystalline lens” by the insertion of cytolyzed fetal tissue (T. A. Sikharuldze, “Exchange of Crystallin Lens in Rabbits by Embryonic Skin Ectoderm,” Bull Acad Sci Georg S.S.R., 14:337 (1956); D. S. Stewart, “Further Observations on Degenerated Crystalline Lenses in Rabbits, with Special Reference to Their Refractive Qualities,” Trans Ophthalmol Soc UK, 80:357 (1960)). Lens fiber differentiation occurs at the equatorial zone, and alpha, beta, and gamma crystallines are produced in proportions similar to fetal or normal lens. The various embodiments of the present invention reaffirm these past findings in the art and demonstrate that regeneration of the lens can be enhanced by restoring lens capsule integrity.

By filling the empty capsular bag with a viscoelastic (Healon® OVD) alone, with air, or perfluoropropane gas, the inventor was able to maintain the capsule tension and prevent folding and adhesion of the capsule, resulting in a more spherical shape to the regenerated lens. Fetal wounds that heal without scar formation have an extracellular matrix that is rich in hyaluronic acid. In the group that received the Healon® OVD alone, regrowth was inconsistent because of scarring of the anterior capsulotomy site to the posterior capsule in some cases, indicating that hyaluronic acid alone was insufficient to account for the enhanced effects noted. The regrowth was much faster and more regular in the air group, whereas the perfluoropropane gas was associated with more scarring of the anterior capsule and delayed lens regeneration because of its slow resorption time. Additionally, lens regrowth proceeded from the periphery along both anterior and posterior capsules surrounding the material filling the capsule bag, which may have caused increased pressure and enhanced cellular elongation and differentiation (A. Gwon et al., “Restoring Lens Capsule Integrity Enhances Lens Regeneration in New Zealand Albino Rabbits and Cats,” J Cataract Refract Surg., 19: 735-746 (1993)).

As discussed hereinabove, lens regeneration has been shown to depend on restoring lens capsule integrity. With the lens capsule as an external scaffold for the lens fibers to differentiate, the regenerated lenses have had a normal cortex but the nucleus has contained a star-shaped opacity related to the irregular growth pattern and misalignment of the earliest lens fibers. The inventor therefore evaluated a high viscosity hyaluronic acid as an internal scaffold to synchronize proliferation in the lens capsule during lens regeneration in rabbits (Example 5). The inventor's study showed that the high viscosity hyaluronic acid provided an internal scaffold for the proliferation and differentiation of lens fibers following endocapsular lens extraction. Moreover, this study demonstrated the beneficial effect that high viscosity hyaluronic acid compositions may have on the enhancement of lens proliferation and differentiation. Compositions of high viscosity hyaluronic acid which have a beneficial effect on lens regeneration obviate the limitations of prior art. For example, the Inventor demonstrated enhancement of lens regeneration in Examples 4 and 5. The inventor demonstrated that hyaluronic acid in the form of Restylane® or Perlane® OVD's may be used to enhance lens regeneration (Example 4).

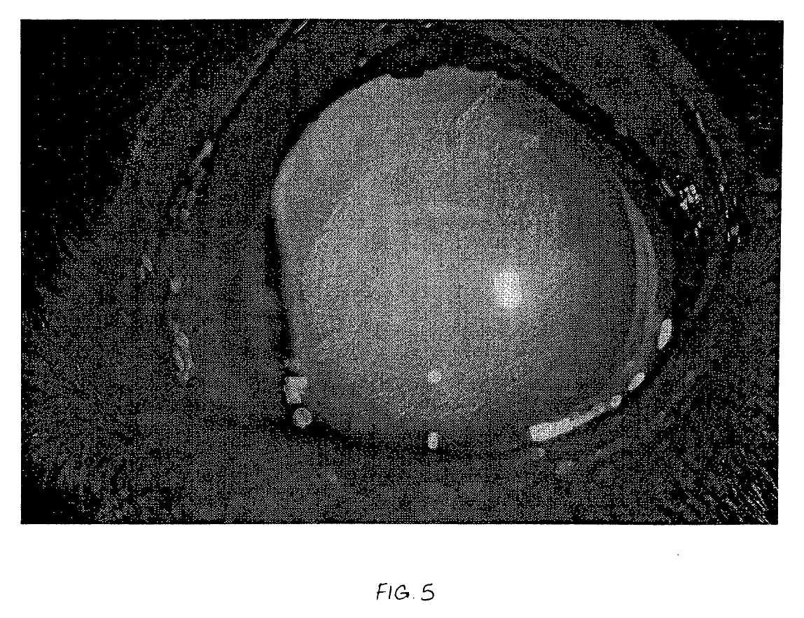

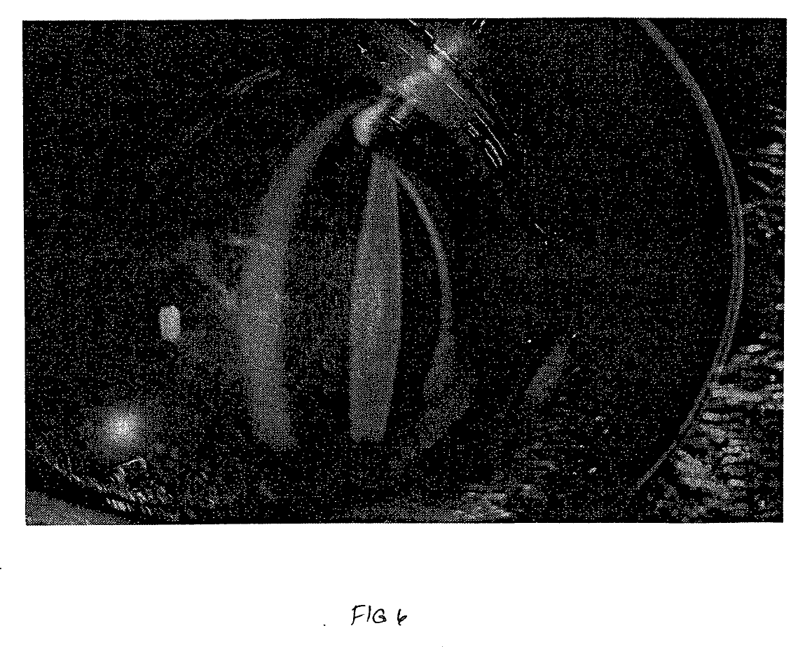

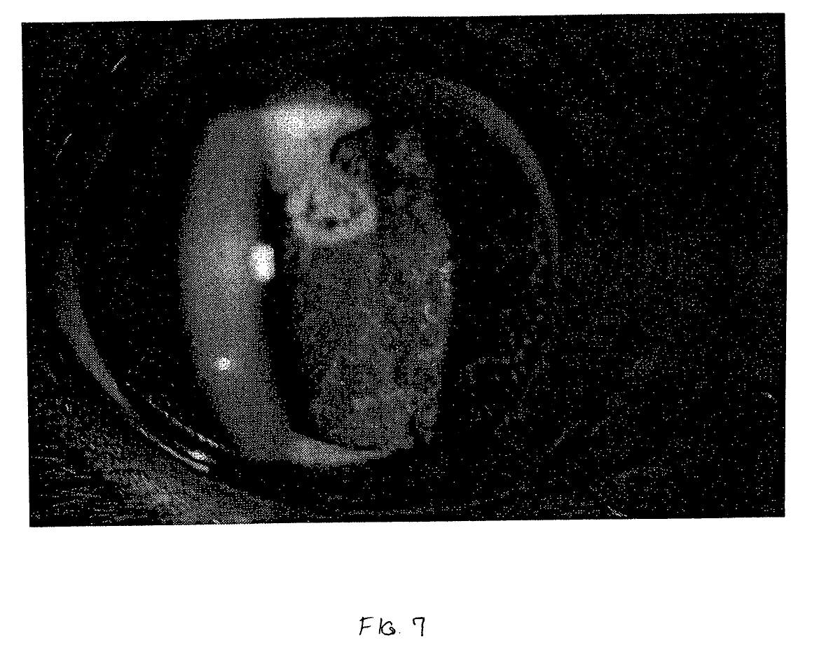

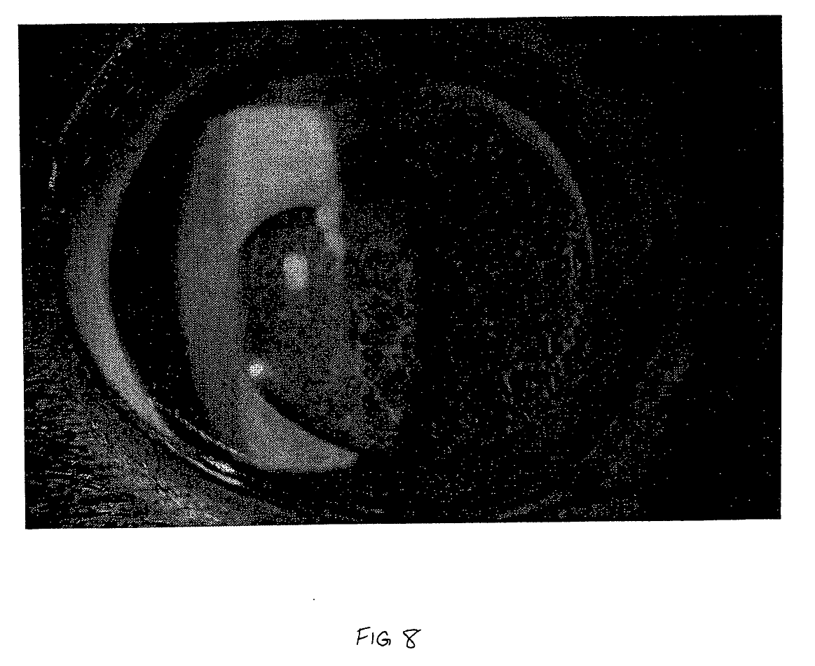

FIG. 5 depicts the honeycomb appearance of Perlane® OVD in the lens capsule bag, and FIG. 6 depicts the clarity of the regenerated lens structure adjacent to the Perlane® OVD in accordance with an embodiment of the present invention. Additionally, FIGS. 7 and 8 depict the honeycomb appearance of the early lens growth enhanced by Restylane® OVD in accordance with an embodiment of the present invention. As seen when comparing FIGS. 5 and 7, Restylane® OVD produces a larger honeycomb pattern in the lens capsule bag than Perlane® OVD.

In past studies, focal laser photoablation of lenticular tissue has been shown to be a relatively safe, noninvasive procedure that can be performed without lens capsule disruption (A. Gwon et al., “Focal Laser Photophacoablation of Normal and Cataractous Lenses in Rabbits: Preliminary Report,” J Cararact Refract Surg., 21:282-286 (1995)). In the Inventor's current study, focal photocoagulation provided limited removal of retained hyaluronic acid. The inventor's findings as discussed in Example 5 support the usefulness of an intralenticular device/therapeutic and its in vivo modification in the treatment of lenticular disorders.

Furthermore, the inventor quantitatively analyzed the clarity of regenerated lens material after endoscapsular lens extraction and restoration of the lens capsular bag with and without implantation of an intracelenticular disc lens (A. Gwon, “Intralenticular Implant Study in Pigmented Rabbits: Opacity Lensmeter Assessment,” J Cataract Refract Surg, 25, 268-277). The inventor sought to provide an internal scaffold for the lens epithelial cells by implanting a semi-permeable synthetic lens (Example 6). The lens epithelial proliferation and differentiation did occur around the intralenticular implant, although the clarity was less than optimal. Insertion of an intralenticular disk lens into the lens capsule bag after endocapsular lens extraction was associated with poor optical quality primarily of the posterior regenerated lens tissue. While not wishing to be bound by any particular theory, it is postulated that the intralenticular lens may have blocked a factor necessary for normal metabolism of the posterior lens fibers or may have allowed the accumulation of a toxic substance behind it.

The present invention relates to the enhancement of lens regeneration though the use of hyaluronic acid and/or a collagen product in the lens capsule to improve lens cell proliferation and differentiation, as for example, to improve the configuration, shape and structure of regenerated lenses.

In one embodiment of the present invention, a viscoelastic substance such as hyaluronic acid may be used for capsule bag filling to enhance the regeneration of lenses following phacoemulsification and irrigation/aspiration of both the natural and cataractous lens and sealing of the anterior capsule. Various quantities, molecular weights, concentrations, and/or forms of hyaluronic acid products may be used to improve the lens cell proliferation and differentiation. For example, a quantity between 0.01 to 3 cc of hyaluronic acid may be used to fill the lens capsule bag to improve the lens cell proliferation and differentiation.

In another embodiment, hyaluronic acid compound at a concentration of about 20 mg/ml in the form of Restylane® OVD, Perlane® OVD or similar formulations (Example 4) may be used to fill the capsule bag to enhance lens regeneration. In the range of effective concentrations, the hyaluronic acid compound will generally be in the form of a gel or similarly viscous composition. Effective concentrations of hyaluronic acid compounds include those that biodegrade or dissolve after about two weeks postoperatively. For example, in one embodiment, an effective concentration of a hyaluronic acid compound dissolves between two to eight weeks postoperatively, as a compound with this property is believed to enhance lens regeneration. Particularly effective concentrations of hyaluronic acid compounds may biodegrade or dissolve about 2-3 weeks postoperatively.

For optimum lens regeneration to take place, the hyaluronic acid compound used to fill the capsule bag should not biodegrade too quickly to give the hyaluronic acid compound ample time to enhance lens regeneration. The hyaluronic acid or viscous substance used in accordance with the present invention should retain the desired shape or provide a scaffold until the earliest lens fibers have measurably aligned to allow normal lens structure and suture formation. The time that the hyaluronic acid compound should remain in the capsule bag before it biodegrades or is degraded depends on the age of the animal because lens regrowth rates vary with age (FIG. 1). For example, because initial lens regrowth rate in old rabbits is slower than in young rabbits (FIG. 1), the hyaluronic acid compound used to fill the capsule bag in older rabbits may be formulated to biodegrade (or may be degraded) at a slower rate than a hyaluronic acid compound formulated for use in younger rabbits. This same concept applies to other animals, such as humans. For hyaluronic acid compounds that biodegrade at slower rates, hyaluronic acid may be removed from the lens capsule bag by focal laser photocoagulation or by chemical dissolution as described more fully below and by other methods known in the art.

Hyaluronic acid is used in accordance with the present invention to enhance lens regeneration by providing an internal scaffold for the proliferation and differentiation of lens cells. One skilled in the art will readily appreciate that a variety of high viscosity hyaluronic acid compositions, glycosaminoglycans (GAG's), and/or formulations thereof may be used in accordance with alternate embodiments of the present invention. For example, suitable hyaluronic compositions may include, but are not limited to the following: Restylane® OVD, Perlane® OVD, a variant formulation of Healon® OVD, and/or compositions that include high viscosity hyaluronic acid forms such as those described in U.S. Pat. Nos. 6,537,795; 6,090,596; 4,764,360; 6,086,597; 6,368,585; and 5,681,825; U.S. Patent Application Publication No. 2002/0018898 (Ser. No. 09/855,923), and in European Patent Application 0760863 B1, all of which are incorporated herein by reference in their entirety as if fully set forth. Any variant formulation or analogous composition of any of the aforementioned hyaluronic compounds and/or GAGs including, but not limited to hyaluronic acid forms with higher or lower molecular weights, hyaluronic acid forms at variant concentrations, chondroitin sulfate, a hyaluronic acid/chondroitin sulfate mixture, combinations of two or more of the abovementioned compositions, and/or combinations of any of the aforementioned compositions with other suitable agents may be used in accordance with alternate embodiments of the invention. Furthermore, inventive compositions may include a hyaluronic acid compound as well as any number of conventional carriers, additives, preservatives, antibiotics, therapeutic agents and the like that are generally formulated in pharmacological compositions of this nature, as will be readily appreciated by those of skill in the art. Such additional elements may, for example, promote the safety and/or efficacy of the inventive compound.

In alternate embodiments, other media can be used individually or in combination to enhance the proliferation and differentiation of lens cells in accordance with the present invention; for instance, amniotic fluid, in vitro fertilization media, growth factors (e.g., BD MATRIGEL™ Basement Membrane Matrix and BD MATRIGEL™ Basement Membrane Matrix High Concentration), and/or other substances that can enhance or control the growth and proliferation of cells will be readily appreciated by one skilled in the art.

Still further embodiments of the present invention include methods for treating cataracts and other ocular diseases with phacoemulsification in connection with the inventive use of hyaluronic acid (i.e., filling the capsule bag with an effective amount of a viscoelastic substance, such as an inventive hyaluronic acid compound).

In another embodiment of the invention, lens regeneration is enhanced by sealing the anterior capsulotomy with one or more collagen patches. Insertion of a collagen patch may be effected during a procedure for treating ocular disease and/or correcting vision impairment, as for example, endocapsular lens extraction surgery. The lens capsule integrity is restored by inserting one or more collagen patches during endocapsular lens extraction surgery to seal the anterior capsulotomy and restore its continuity, which thereby improves the shape and structure of the regenerated lenses. It will be appreciated by those skilled in the art that a variety of collagen patches may be used and that the sealing of the capsulotomy may occur in various regions in connection with various embodiments of the present invention. For example, a collagen patch that is composed of bovine collagen type IV or a 24 hour collagen shield (Chiron Ophthalmics, Emeryville, Calif., U.S.A) may be used in accordance with an embodiment of the present invention. Additionally, a collagen patch may be used to seal any opening in the lens capsule bag, not just the anterior capsulotomy. Furthermore, in an alternate embodiment, injectable collagen may be used as a supplement to or a replacement for the inserted collagen patch to further enhance lens regeneration.

In an additional embodiment, collagen may be used as an internal scaffold for lens fiber cell proliferation and differentiation. A variety of collagen-based products may be used, as for example, 25% or 50% suspensions of purified bovine dermis in saline with 0.3% lidocaine (available under the trade name Zyderm I® and Zyderm II® from INAMED Corporation; Santa Barbara, Calif.), monomolecular bovine collagen suspended in solution at 3.5% and 6.5% concentrations (available under the trade name Resoplast® from Rofil Medical International; Breda, Holland), human collagen preparation comprised predominantly of intact collagen fibers as well as other matrix proteins suspended in a neutral pH buffer (available under the trade name Dermalogen® from Collagenesis Corporation; Beverly, Mass.), and/or acellular human dermal graft processed from tissue bank-derived skin (available under the trade name Alloderm® from LifeCell Corporation; Palo Alto, Calif.). The lens capsule is a basement membrane structure primarily composed of collagen type IV and glycosaminoglycans. It acts as an external scaffold for lens epithelial cell differentiation. Collagen has been shown to be advantageous for normal epithelial cell proliferation and differentiation, and it may function in the present invention as an internal scaffold for lens cell differentiation in the capsule bag.

It will be appreciated by those skilled in the art that a variety of collagen types may be used in accordance with the present invention. For example, the collagen patches and other collagen based products used in accordance with the present invention may be derived from bovine, human or synthetic sources; include type IV collagen; include a GAG-based compound or copolymer; and/or include collagen produced by amnion as described in U.S. Patent Application Publication No. 2004/0048796 (Ser. No. 10/397,867) which is incorporated by reference in its entirety as if fully set forth.

In another embodiment, lenticular tissue may be engineered using focal laser photophacocoagulation to remove excess viscoelastic substances and/or modify structure and clarity of the regenerated lens and/or bilenticular lens. As described in U.S. Pat. No. 6,322,556 and U.S. Patent Application Publication No. 2002/0103478 (Ser. No. 09/953,121), which are both incorporated herein by reference as if fully set forth, laser photophacoablation (laser photoablation) has been used to partially remove ocular tissue (e.g., lens tissue) to correct vision deficiencies and to treat other vision-impairing ocular problems without causing substantial damage to the surrounding tissue regions. In the present invention, laser photophacoablation may be used to remove retained high viscosity or viscoelastic substances in the regenerated lens in combination with the inventive use of hyaluronic acid.

Suitable forms of hyaluronic acid that may be used in accordance with the present invention, as for example, Restylane® and Perlane® OVD's, may contain a crosslinked form of hyaluronic acid which is not readily resorbed by the body or lens. To assist in the removal of crosslinked hyaluronic acid in the regenerated lens, focal laser photocoagulation may be performed to remove some of the retained hyaluronic acid. For example, in one embodiment, focal laser photocoagulation may be performed with a Q-switched neodymium: YAG (Nd:Yag) laser over multiple treatment times. In an alternate embodiment, a neodymium: YLF (Nd:YLF) laser or a femtosecond laser may be used in accordance with the present invention. In a further embodiment, focal laser photocoagulation may be performed with a femtosecond laser in accordance with the present invention. Alternatively, the hyaluronic acid may be chemically broken down, for example by hyaluronidase.

In an embodiment of the invention, laser photoablation of the retained viscous material in the lens capsule bag may be performed by directing a pulsed laser beam at the viscous material with an amount of energy effective for photoablating the viscous material without causing substantial damage to the surrounding tissue region. The laser is initially directed, or focused, at a focal point below an anterior surface of the viscous material, and such focal point is moved toward the anterior surface of the viscous material in order to ablate the viscous material. The anterior surface is in reference to the lens capsule bag in which the viscous material is retained. An alternative embodiment of the present invention includes the initiation of photoablation of the surface of the viscous material's anterior surface and thereafter moving the focal point inwardly and away from the anterior surface in order to promote the absorption of laser by products by adjacent healthy tissue. In other embodiments, a plurality of portions of the viscous material may be photoablated. Any of the foregoing variations of laser photoablation methods may be performed multiple times until the desired quantity of viscous material is removed.

Various laser types, laser characteristics, and various methods for laser photophacoablation, are described in U.S. Pat. No. 6,322,556 and U.S. Patent Application Publication No. 2002/0103478 (Ser. No. 09/953,121), both of which are incorporated by reference herein as if fully set forth, may be used in accordance with the present invention. Additionally, it will be readily appreciated by those skilled in the art that a variety of other lasers and other methods for laser photophacoablation may be used in accordance with alternates embodiments of the present invention.

The laser used in the present invention may have a variety of characteristics. For example, the laser used may have any or all of the following characteristics: an operating frequency in the visible and infrared (IR) spectrum; a repetition rate ranging from about one to about 1000 Hertz; a pulse width ranging from about 1 femtosecond to about 1 millisecond; an energy level per pulse ranging from about 1 nanojoule to about 50 millijoules; a focused spot size (diameter) between about 1 micron and about 100 microns; a “zone of effect limited to between about 1 and about 200 microns with little collateral effect. In one embodiment, the laser may have the following characteristics: an operating frequency of about 1053 nanometers (nm); a repetition of about 1000 pulses per second; a pulse width of about 60 picoseconds; an energy level per pulse of about 60-140 microjoules, a focused spot size (diameter) of about 20 microns, and a zone of effect limited to about 50 microns.

In one aspect, the present invention teaches the administration of hyaluronidase to dissolve the retained hyaluronic acid following endocapsular lens extraction. Following administration of the hyaluronidase, the regenerating lens tended to collapse centrally following the intralenticular hyaluronidase injection. As lens regeneration progressed the irregular alignment of central fibers resulted in a spherical regenerated lens with a nuclear star shape opacity of poor clarity and a fairly normal cortex with good structure and clarity.

In those embodiments of the present invention directed to methods for treating ocular disease and/or correcting vision impairment, one can use these methods to treat any disease in which enhancing lens regeneration has a beneficial effect on a patient (e.g., ameliorating a disease, lessening the severity of its complications, preventing it from manifesting, preventing it from recurring, merely preventing it from worsening, or a therapeutic effort to effect any of the aforementioned, even if such therapeutic effort is ultimately unsuccessful). Methods of the present invention may be used to treat any diseases which are affected by lens tissue loss or damage, or ocular conditions or impairments which involve a medical procedure comprising the removal or alteration of lens tissue.

### EXAMPLES

The examples described herein demonstrate various aspects of the present invention in connection with the enhancement of lens regeneration. Such uses may be particularly advantageous in treating those diseases in which regenerating lens cells has a beneficial effect. Such diseases include, for example, cataracts. However, as noted above, the present invention has uses beyond those illustrated herein, and the ensuing examples are in no way intended to delineate the extent to which the present invention may find application in the medical arts.

### Example 1

### Preparation of Animals

Endocapsular lens extraction by phacoemulsification and irrigation/aspiration of the lens through a 2-3 mm capsulorhexis was performed in rabbits. Following removal of the lens, a high viscosity hyaluronic acid was injected into the capsule bag, and a collagen patch was placed inside the capsulorhexis and brought up against the capsulotomy with an air bubble in the bag. The animals were followed postoperatively by slit lamp biomicroscopy. Following euthanasia, eyes were enucleated, paraffin embedded and H & E slides prepared for routine histology.

### Example 2

### Insertion of Collagen Patch in Lens Capsule

The inventor restored the lens capsule integrity by inserting a collagen patch at the time of endocapsular lens extraction surgery to seal the anterior capsulotomy and to improve the shape and structure of the regenerated lenses. Lens regeneration was first noted as early as one to two weeks following surgery. Regenerated lens filled approximately 50% of the capsule bag at two weeks and 100% by five weeks. Subsequent growth was in the anterior-posterior direction. Lens thickness increased by 0.3 mm per month. The regenerated lenses were spherical with normal cortical structure and a nuclear opacity. Restoration of lens capsular integrity with a collagen patch following endocapsular lens extraction enhanced the shape, structure, and growth rate of the regenerated lenses. In addition, lens regeneration was shown to occur in two cats.

### Example 3

### Filling of Capsule Bag with Air, Sodium Hyaluronate, or Perfluoropropane

After insertion of a collagen patch the inventor then filled the capsule bag with air, sodium hyaluronate (Healon® OVD), or perfluoropropane gas to prevent adhesions between the anterior and posterior capsules and to maintain capsule tautness and shape. Lens thickness measurements at one and two months represent the filled capsule bag containing lens regrowth as well as most probably a mixture of balanced salt solution (BSS), Healon® OVD, aqueous, and collagen degradation products and injured lens epithelial cells. From 4 to 12 months, lens thickness increased gradually but in progressively smaller increments.

The collagen patch occluded the anterior capsulotomy for up to two weeks before dissolution resulting in a linear scar at the capsulotomy site. The capsule bag was distended and maintained taut without surface wrinkles for one, five, and eight weeks in the Healon® OVD, air, and perfluoro-propane groups, respectively.

Lens regrowth was noted as early as one, two, and three weeks in the Healon® OVD, air, and perfluoropropane groups, respectively. Regrowth proceeded from the periphery of the capsule bag centrally along the anterior and posterior capsules, engulfing the Healon® OVD/aqueous. Lens regrowth was more complete, and the overall shape of the lens was spherical in the air and perfluoropropane groups. With time, the earliest fibers became progressively more compacted centrally, resulting in a star-shaped nuclear opacity. In all groups, the newer cortical fibers appeared translucent and similar to normal, with the air group having the smallest nuclear opacity and the perfluoropropane group having the largest.

### Example 4

### Filling of Capsule Bag with Hyaluronic Acid, 20 mg/ml, in the Form of Restylane® and Perlane® OVD's

Hyaluronic acid, 20 mg/ml (in the form of Restylane® and Perlane®) was shown to enhance lens regeneration with the new lens cells differentiating in a normal configuration around the retained form of hyaluronic acid (FIGS. 5-8). The first signs of lens cell proliferation were seen as early as two weeks postoperative with full growth around the centrally retained hyaluronic acid at seven weeks. The newly formed lens had normal clarity and shape peripheral to the retained Restylane® OVD or Perlane® OVD in the center. While the capsule bag acted as an outer scaffold for the differentiation of lens fibers, the hyaluronic acid appeared to act as an inner scaffold for the lens fiber differentiation. Restylane® and Perlane® OVD's were shown to promote lens fiber alignment such that the innermost regenerated lens fibers, as well as the outermost cortical lens fibers formed an elliptical or spherical shaped lens.

### Example 5

### Evaluation of Hyaluronic Acid as an Internal Scaffold for the Proliferation and Differentiation of Lens Fibers

The inventor evaluated a high viscosity hyaluronic acid as an internal scaffold to synchronize proliferation in the lens capsule during lens regeneration in rabbits. Endocapsular lens extraction of the lens through a 2-3 mm capsulorrhexis was performed in 8 eyes of 4 Dutch Belt pigmented rabbits (age 8 weeks, wt 2 kg). Healon® OVD was injected into the capsule bag. A collagen patch was placed inside the capsulorrhexis and brought up against the capsulotomy with an air bubble in the bag. The animals were assessed postoperatively by slit lamp biomicroscopy. Following euthanasia, eyes were enucleated, parafin-embedded and H & E slides prepared. In one eye, focal laser photocoagulation was performed with an Nd:YAG laser to remove some of the retained hyaluronic acid.

In 3 eyes, the collagen patch slipped and capsulotomy closure was incomplete. In 5 eyes, lens cellular proliferation was noted at 2 weeks postop and full lens growth around the central viscoelastic mass was noted at 7 weeks. The regenerated lenses had a normal spherical shape, good clarity, and lens structure with normal fiber alignment around the residual viscoelastic material. Histology revealed normal lens structure with a monolayer of anterior lens epithelium, lens differentiation at the equatorial region, and normal lens fiber morphology. Centrally, the retained hyaluronic acid appeared as an elliptical homogenous bluish mass. In the eye treated with focal photocoagulation, partial clearing of the hyaluronic acid was noted.

High viscosity hyaluronic acid provided an internal scaffold for the proliferation and differentiation of lens fibers following endocapsular lens extraction in Dutch Belt pigmented rabbits. Focal photocoagulation provided limited removal of retained hyaluronic acid. The data support the utility of an intralenticular device/therapeutic and its in vivo modification in the treatment of lenticular disorders.

### Example 6

### Opacity Lensmeter Assessment

The clarity of regenerated lens material was quantitatively analyzed.

Endocapsular lens extraction through a 2-3 mm capsulorrhexis was performed in New Zealand/Dutch Belt pigmented rabbits. In the test group, an Acuvue® or a Survue® disposable contact lens was intralenticularly implanted in both eyes with the aid of Healon® OVD, and in the control group, the capsular bags of both eyes were distended with Healon® OVD only and no artificial lens was implanted in either eye. A 24 hour collagen shield (Chiron Ophthalmics) was cut freehand to approximately 2 to 3 times the anterior capsulotomy diameter, which ranged from 2.5 to 3.5 mm after lens removal. The collagen patch was coated with Healon® OVD and inserted into the capsule bag in both test and control eyes. Air was injected to distend the capsular bag and maintain the collagen patch behind the anterior capsulotomy. The animals were assessed postoperatively by slit lamp biomicroscopy. The Interzeag Opacity Lensmeter 701 (OLM) was used to quantify lens opacification. Following euthanasia, eyes were examined by light microscopy, paraffin-embedded and H & E slides prepared, and prepared for electron microscopy examination.

Mean OLM results were similar in both groups at weeks 1, 2, 3 and 4. After one month, progressive central compaction of early irregular regenerated lens fibers was associated with increased OLM readings that were higher in the intralenticular implant group that in the control group. Regenerated lens opacification was greater in tissue posterior to the intralenticular lens than anterior to the disc lens.

In the early postoperative period, an OLM score less than 20 generally reflected the clear media before full lens regenerative tissue filled the capsular bag. As time progressed, the earliest imperfectly aligned regenerated lens fibers became progressively more compacted centrally resulting in a dense star-shaped nuclear opacity and increasing OLM score.

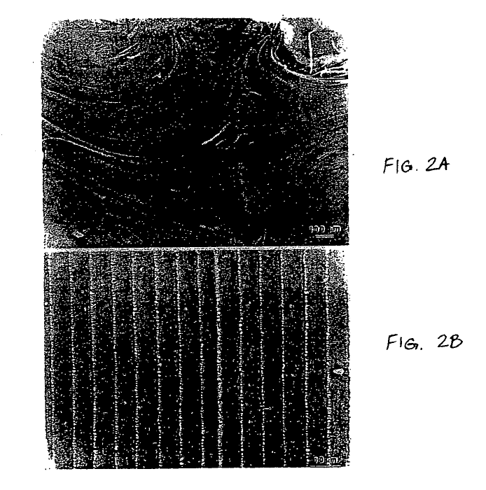

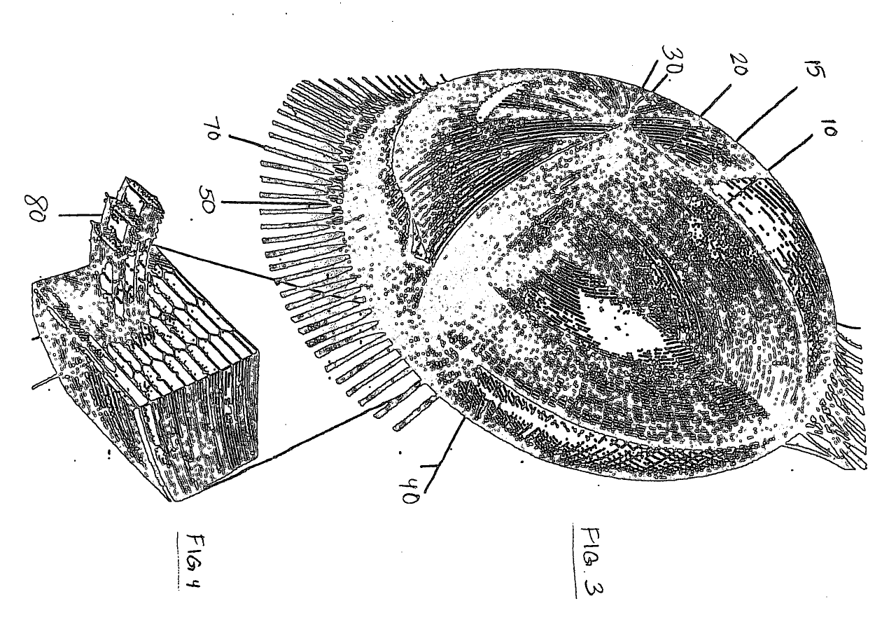

Additionally, upon gross examination, it was apparent that relatively normal (uniform size and shape) fibers grew around the intralenticular implant. However, on close scanning electron microscopic examination of fiber arrangements, fiber ends did not overlap and form normal sutures (FIG. 2A). These fibers were not grouped into quadrants. The ends of these fibers were acutely curved in varying directions. The result was the creation of swirling suture patterns with more than 2 anterior and posterior branches. Nevertheless, the fibers were clearly arranged in concentric shells and radial cell columns. In addition, fibers were uniformly hexagonal along their length and featured typical lateral interdigitations arrayed along their length (FIG. 2B).

### Example 7

### Use of Hyaluronidase/Hyaluronic Acid in the Rabbit Lens Regeneration Model

Hyaluronidase was administered intralenticularly into the retained Restylane OVD mass at one week (6 days) and one month (28 days) to evaluate the dissolution of the retained Restylane in modulating the lens regenerative process following endocapsular lens regeneration in 5 month old NZA rabbits weighing 3.0 kg.

Immediately postoperative, the anterior capsulotomy was sealed by a collagen patch for up to 2-3 weeks after which the anterior capsulotomy was noted to be closed by a thin scar. The capsule bag was initially distended by the Restylane in all eyes. Following the Vitrase injection at day 6, the capsule bag was noted to be flat in 2 of the 3 eyes receiving the intralenticular injection and as lens regrowth progressed the capsule bag had a normal spherical shape except in one eye with an adhesion at the capsulotomy site. In the one month group, the regenerated lens remained spherical through day 91.

New lens regrowth filling 5 to 20% of the capsule bag or retained cortical material was first noted at day 6 in the one week group and at day 27 in the one month group. Lens regrowth gradually progressed in both groups. Full lens regrowth was first noted at day 35 in 1 of the one-week eyes and at day 41 in all eyes.

Initially lens regrowth progressed from the periphery toward the center fairly uniformly around the Restylane OVD center. Following intralenticular Vitrase injection, the regenerating lens collapsed centrally with irregular alignment of lens fibers giving the nucleus a faint star shape. Lens nuclear clarity was slightly better in the one week group compared to the one month group. The lens cortex had good clarity and structure with occasional vacuoles and/or retained Restylane OVD in both groups.

A total of three—New Zealand white rabbits 5 months old were used for this study. Hyaluronidase in the form of Vitrase was injected intralenticularly at one week and one month following endocapsular lens extraction. A 24 hour PROSHIELD Collagen Corneal Shield to seal the capsulotomy was placed; Restylane® OVD was instilled as an internal scaffold for the lens epithelial cells to proliferate and differentiate. It is noted that other collagen shields will also be suitable for use with the present invention.

Surgery: Aphakic Implantation

Rabbits (N=3, New Zealand white) were anesthetized. The surgical eye was dilated, eyelashes were trimmed, and the ocular area was disinfected using standard techniques. A wire lid speculum was inserted to retract the lids, and a corneal incision was made with a 2.85 mm keratome. Healon GV OVD (Advanced Medical Optics, Santa Ana, Calif.) was injected to maintain anterior chamber depth and an approximately 2-3 mm continuous curvilinear capsulorrhexis was performed. A 21 gauge phacoemulsification tip was inserted through the corneal wound and endocapsular lens extraction was performed by phacoemulsification and irrigation/aspiration with BSS (no heparin or epinephrine was used). Considerable care was taken to remove all lens cortical material by diligent irrigation and aspiration. The 24 hour PROSHIELD Collagen Corneal Shield (Alcon Laboratories, Inc, Fort Worth, Tex.) was cut freehand to approximately 2-3 times the size of the capsulotomy. The customized collagen patch was moistened and inserted into the capsule bag. A lens hook was used to maneuver the patch behind the anterior capsulotomy with at least a 1 mm overlap internally. The Restylane OVD (0.03 cc) (Q-Med Scandinavia, Inc., Princeton, N.J.) was injected into the capsule bag followed by an air bubble to stabilize the patch against the capsule. At the end of each surgery standard antibacterial agents were applied.

The hyaluronidase was received in a single-use glass vial, Lyophilized, Ovine 6200 USP units kept refrigerated. While the source of hyaluronidase is not considered to be important, Vitrase (Distributed by: ISTA Pharmaceuticals, Inc., Irvine, Calif. Manufactured by: Cardinal Health, Albuquerque, N. Mex.), Lyophilized, Ovine, 6200 USP Units Single—Use Vial (Nonpreserved) is suitable for the present invention. Prior to intralenticular injection, bench top testing was performed to confirm activity of the hyaluronidase.

Vitrase (310 units/ml) was injected intralenticularly in three of the rabbit eyes. Rabbit eye 73268 OS received 0.08 ml (˜21 units) Vitrase intralenticularly into the Restylane mass and rabbit eyes 73267 OS and 73269 OS received 0.03 ml (9.3 units) Vitrase intralenticularly into the Restylane OVD mass using a 30 g needle. A slight amount of the Vitrase was noted to extrude from the capsule bag on withdrawing the needle. It is noted that “OS” refers to a left eye, while “OD” refers to a right eye.

The second group of three 5-month old rabbit eyes received Vitrase (310 units/ml) intralenticularly twenty-two days later. Rabbit eye 73268 OD received 0.04 ml (˜13 units) Vitrase and Rabbit eyes 73267 OD and 73269 OD received 0.03 ml (˜9.3 units) Vitrase intralenticularly into the Restylane mass using a 30 g needle. Again, a slight amount of the Vitrase was noted to extrude from the capsule bag on withdrawing the needle.

Phacoemulsification and Irrigation/Aspiration was performed with plain BSS with no additive. Mean capsulorrhexis size was 2.4±0.6 mm in the one week group and 2.0±0.5 mm in the one month group. 0.03 cc Restylane was injected into the capsule bag following endocapsular lens extraction. Air was injected into the capsule bag following placement of the customized collagen patch.

Slit Lamp Biomicroscopy

Inflammation

Immediately postoperative all wounds were intact. Trace conjunctival injection resolved by day 6 and trace to mild corneal edema/haze resolved by day 8. Anterior chamber cells and flare were not noted. Mild to moderate anterior chamber fibrin was seen in all eyes and resolved by day 6.

Posterior Synechiae

Trace posterior synechiae adjacent to the capsulorrhexis was first noted at day 1 in all eyes and persisted in a few eyes through day 41 in the one month group and day 69 in the one week group.

Capsulotomy

Mean anterior capsulorrhexis size was 2.0±0.5 mm in the one month group and 2.4±0.6 mm in the one week group. Immediately postoperative, the anterior capsulotomy was sealed by a collagen patch in all eyes. The collagen patch remained visible for approximately 2-3 weeks after which the anterior capsulotomy was noted to be closed by a thin scar, except in 1 of the 3 one week eyes where the anterior capsulotomy sealed to the posterior capsule and the capsule bag remained constricted throughout the study. Such an occurrence may be caused by the collagen patch slipping, the bag collapsing, or if too much fibrin formation occurs. Two months (day 91 postoperative) following the intralenticular Vitrase injection at one month, the capsulotomy was noted to be sealed to the posterior capsule in 1 eye.

Capsule Bag/Lens Shape

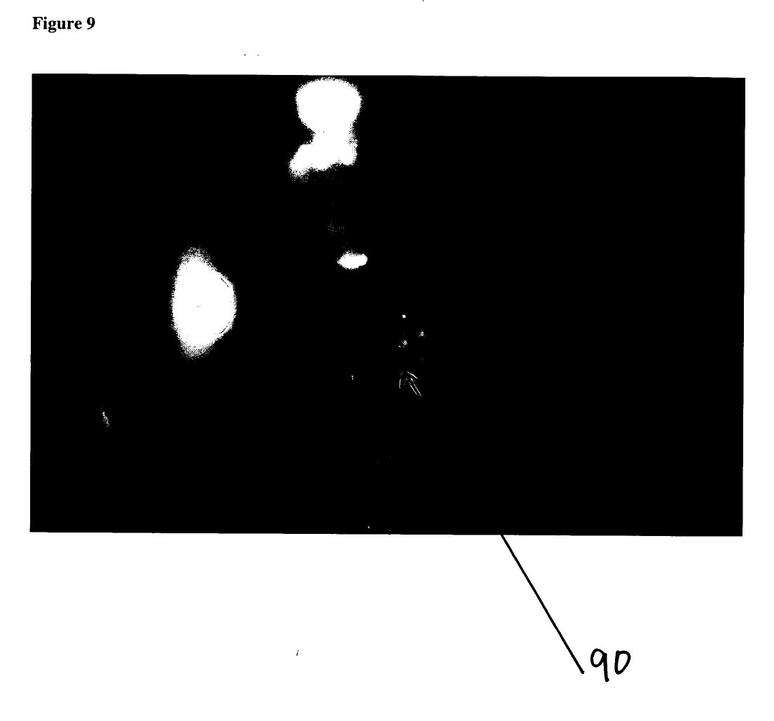

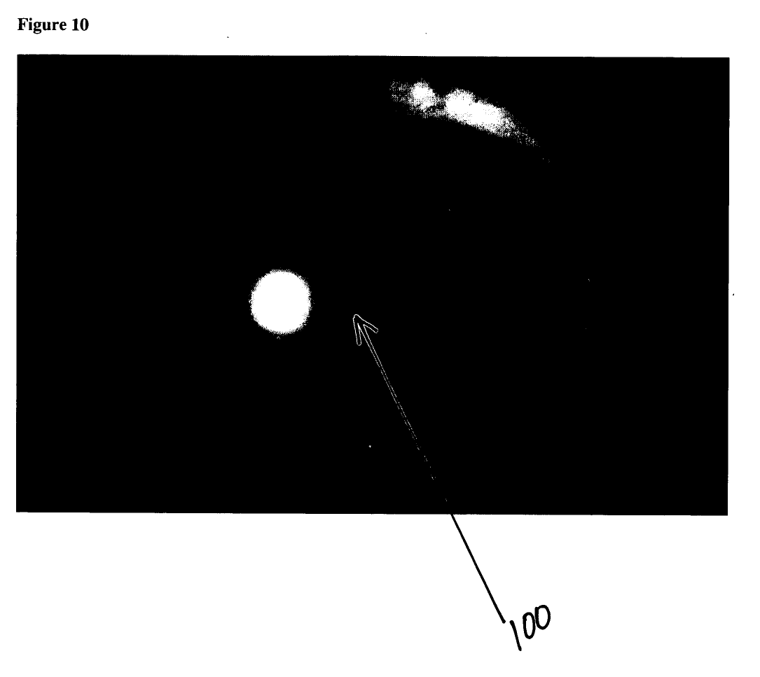

Immediately postoperative, the capsulotomy was sealed by a collagen patch and the capsule bag was distended with Restylane which had a cobblestone appearance in 6 of 6 eyes. See FIG. 9, showing day 6 pre-Vitrase intralenticular injection. The collagen patch is in place, the capsulotomy closed and the cobblestone appearance of Restylane 90 is visible.

Following the Vitrase injection at day 6, the capsule bag was noted to be flat in 2 of the 3 eyes receiving the intralenticular injection. In the 3 untreated eyes, the capsule bag was distended centrally and flat in the periphery. As lens regrowth progressed the capsule bag had a normal spherical shape in 2 of 3 eyes in the one week group. The remaining eye the regenerated lens was slightly constricted. In the one month group, the regenerated lens remained spherical through day 91.

Restylane in Capsular Bag/Vitrase Injection

The left eye of each rabbit received Vitrase intralenticularly into the Restylane mass on day 6. One day post injection of the Vitrase, the Restylane appeared to be dissolved and a clear fluid filled area 100 was noted in the center of the regenerating lens (FIG. 10).

On day 28 the right eye of each rabbit received Vitrase intralenticularly into the Retylane mass. One day post injection of the Vitrase, the Restylane appeared to be dissolved and a clear fluid filled area was noted in the center of the regenerating lens.

Progression of Lens Regeneration

New lens regrowth filling 5 to 20% of the capsule bag or retained cortical material was first noted at day 6 in the one week group and at day 27 in the one month group. Lens regrowth gradually progressed in both groups. Full lens regrowth was first noted at day 35 in 1 of the one-week eyes and at day 41 in all eyes.

Initially lens regrowth progressed from the periphery toward the center fairly uniformly around the Restylane center. Following intralenticular Vitrase injection, the regenerating lens collapsed centrally with irregular alignment of lens fibers giving the nucleus a hazy star shape or a swirl pattern in lens constricted by adhesions at the capsulotomy site (73269os). Lens nuclear clarity was only slightly better in the one week group compared to the one month group.

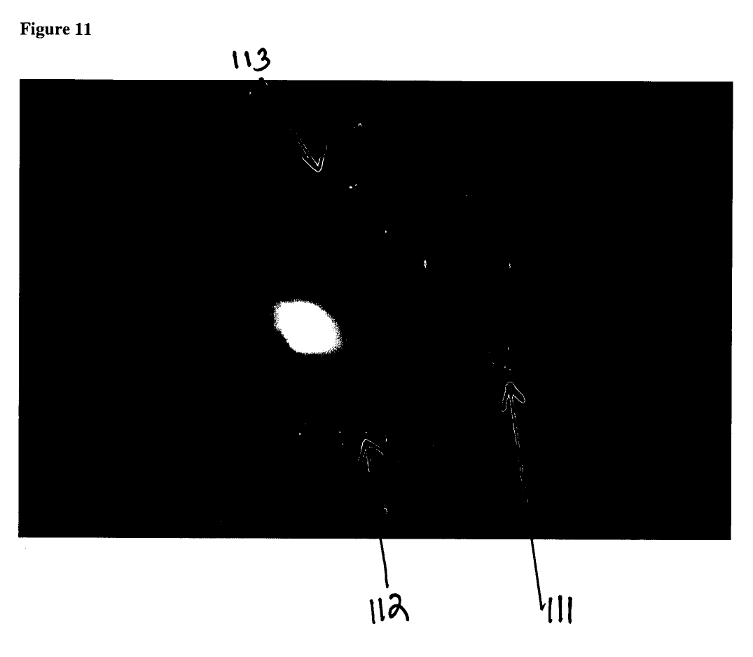

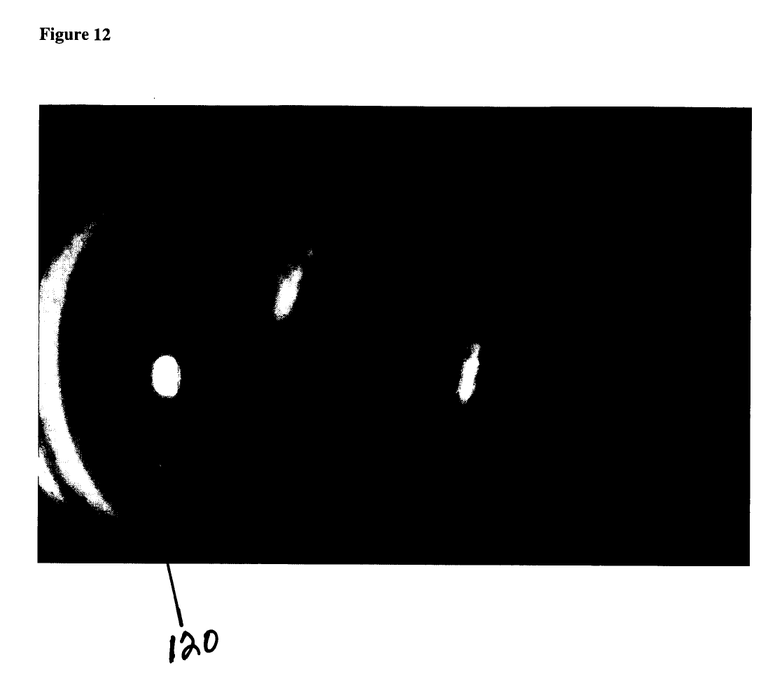

The lens cortex for both the one-week and one-month groups had good clarity and structure with occasional vacuoles and/or retained Restylane. See FIG. 11, which represents one of the regenerated lenses from the 1-week group. As may be seen, the cortex 111 has a good structure, with a few vacuoles/retained Restylane 112 and a lacy nuclear star opacity 113. Similarly, FIG. 12 shows a spherical regenerated lens from the one month group. The cortex 120 has good structure and the capsulotomy sealed to linear scar.

Lens Regrowth (Vitrase Injection Day 6-OS)

Hyaluronidase was effective in dissolving retained Restylane following endocapsular lens extraction in 5 month old NZ white rabbits. The regenerating lens tended to collapse centrally following the intralenticular hyaluronidase injection. As lens regeneration progressed the irregular alignment of central fibers resulted in a spherical regenerated lens with a nuclear star shape opacity of poor clarity and a fairly normal cortex with good structure and clarity.

### Example 8

### Use of Hyaluronidase/hyaluronic acid in the Rabbit Lens Regeneration Model

Tests similar to Example 7, above, were conducted. In this example, hyaluronidase was administered intralenticularly into the retained Restylane OVD mass at 12 days (the two week group) and 21 days to evaluate the dissolution of the retained Restylane in modulating the lens regenerative process.

New lens regrowth filling 5 to 50% of the capsule bag was first noted at day 20 in the Vitrase 2 week group and at Day 7 in the Vitrase 3 week group. One day post injection of the Vitrase, the Restylane appeared to be dissolved and a clear fluid filled area was noted in the center of the regenerating lens.

The above examples demonstrate that the optimum time for Vitrase injection in rabbit eyes is approximately within the 1-6 weeks following lens extraction and intralenticular injection of a polymer, for example hyaluronic acid. Based on this data, it will be understood by one of ordinary skill in the art that a juvenile human eye will take up to three months to demonstrate the appropriate amount of lens regeneration, and an adult human eye will take even longer (up to 6 months, 9 months or, in some cases, possibly even a year). The appropriate time for Vitrase injection is best determined by the amount of lens growth seen in the eye. Specifically, Vitrase should be injected once a ring of lens tissue may clearly be envisioned, defining what will become the outer periphery of the regenerated lens. Generally, such a ring occurs when about 5-20% of lens growth is seen in the peripheral capsule bag by slit lamp examination (or other means of assessment). In any event, the hyaluronidase should be injected prior to the regeneration of 70% of the lens.

The rate of the growth may be determined, in part, by residual tissue that is left in the bag at the time of surgery. This likely accounts for the wide variation seen in lens regeneration time, above.

### Example 9

### Use of hyaluronic acid in the Rabbit Lens Regeneration Model

Tests similar to Examples 7 and 8, above, were conducted. This study was designed to evaluate the role of hyaluronidase (which neutralizes native hyaluronic acid produced by lens epithelial cells) in modulating the lens regenerative process compared to hyaluronic acid when placed into the capsule following endocapsular lens extraction. Following insertion of the collagen patch, Restylane® (0.03 cc) was injected in one group of eyes, while Vitrase (0.03 cc-9.3 units) was injected into a second group of eyes.

New lens regrowth filling 5% of the capsule bag was first noted at day 20 in both groups. Lens regrowth gradually progressed in both groups until it filled 100% of the capsule bag in the Vitrase group and 70% of the capsule bag in the Restylane® OVD group at day 56. In the latter group, the Restylane® OVD was retained centrally in the capsule bag.

In the Vitrase group, the capsule bag was distended with clear fluid which became progressively hazy to milky as lens regrowth progressed centrally in a non uniform pattern. The fully regenerated lens was spherical with an irregularly shaped hazy nucleus and a normal cortex with fairly good clarity and structure. Without intending to be bound by theory, it is believe that the hazy nucleus was due to abnormal alignment of the earliest lens fibers.

In the Restylane® OVD group, the capsule bag was distended by the cobblestone appearance of the Restylane® OVD which became progressively compacted centrally as lens regrowth progressed from the periphery in a fairly uniform manner around the Restylane® OVD center. The fully regenerated lens was spherical, which a spherical nucleus of hazy Restylane, and a normal cortex with good clarity and structure.

It was noted that more vacuoles were present in the Vitrase group compared to the Restylane® group.

Based on the above findings, it was determined that the regenerated lens receiving hyaluronidase was associated with less clarity, greater vacuoles and a hazy irregularly shaped nucleus as compared to the Restylane enhanced regenerated lens.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for enhancing regeneration of lens cells in a mammal, comprising:
filling a lens capsule bag of said mammal with a composition comprising hyaluronic acid, and
after formation of a ring of lens tissue, administering hyaluronidase into the capsular bag.

2. The method of claim 1, further comprising:
inserting at least one collagen patch in said lens capsule bag.

3. The method of claim 1, wherein said composition is selected from the group consisting of Restylane®, Perlane®, and combinations thereof.

4. The method of claim 1, wherein said hyaluronic acid compound is present in said composition at a concentration of at least about 20 mg/ml.

5. The method of claim 1, further comprising:
phacoemulsifying a lens contained in said lens capsule bag prior to said inserting of said at least one collagen patch.

6. The method of claim 1, wherein the hyaluronidase is administered between 1 week and 6 months after the filling of the capsule bag.

7. A method for treating an ocular condition, comprising:
providing a first composition comprising hyaluronic acid;
inserting said first composition into a lens capsule bag to enhance regeneration of lens cells, thereby treating said ocular condition; and
after formation of a ring of lens tissue, inserting a second composition into the lens capsule bag to dissolve the first composition.

8. The method of claim 7, further comprising:
inserting a collagen patch into said lens capsule bag.

9. The method of claim 7, wherein said first composition comprises hyaluronic acid at a concentration of at least about 20 mg/ml.

10. The method of claim 7, wherein said first composition comprises a crosslinked hyaluronic acid.

11. The method of claim 7, wherein the second composition comprises hyaluronidase.

12. The method of claim 7, wherein said ocular condition is a cataract.

13. The method of claim 7, wherein the hyaluronidase is administered between 1 week and 6 months after the inserting of the first composition into the capsule bag.

14. A method for enhancing regeneration of lens cells in a mammal, comprising:
providing a polymeric composition;
filling a lens capsule bag of said mammal with said composition, and
administering an enzymatic compound into the lens capsule bag.

15. The method of claim 14, wherein the polymeric compound is hyaluronic acid.

16. The method of claim 14, wherein the enzymatic compound is hyaluronidase.

17. A method for enhancing regeneration of lens cells in a mammal, comprising:
filling a lens capsule bag of said mammal with a composition comprising hyaluronic acid,
waiting a period of time sufficient for formation of a ring of lens tissue; and
administering hyaluronidase into the capsular bag.

18. The method of claim 17, further comprising:
inserting a collagen patch into said lens capsule bag.

19. The method of claim 17, wherein said first composition comprises hyaluronic acid.

20. The method of claim 17, wherein said first composition comprises a cross-linked hyaluronic acid.

21. The method of claim 17, wherein the second composition comprises hyaluronidase.

22. The method of claim 17, wherein the waiting period is less than 6 months.

23. The method of claim 17, wherein the waiting period is between 1 week and 6 months.
